(19) United States
(12) Patent Application Publication
(10) Pub. No.: US 2013/0237815 A1
(43) Pub. Date: Sep. 12, 2013

(54) METHOD FOR DETERMINING A FOUR-DIMENSIONAL ANGIOGRAPHY DATASET DESCRIBING THE FLOW OF CONTRAST AGENT

(71) Applicant: Klaus Klingenbeck, Aufsess (DE)

(72) Inventor: Klaus Klingenbeck, Aufsess (DE)

(21) Appl. No.: 13/784,914

(22) Filed: Mar. 5, 2013

(30) Foreign Application Priority Data
Mar. 9, 2012 (DE) 102012203751.9

(51) Int. Cl. A61B 6/00

(52) U.S. Cl. CPC A61B 6/504; A61B 6/481; A61B 6/4441; A61B 6/5235; A61B 6/501
USPC 600/431

(57) ABSTRACT

A method for determining a four-dimensional angiography dataset describing the flow of contrast agent over time through a blood vessel system of the body of a patient is provided. Four-dimensional flow information is obtained from two-dimensional images captured in a capture time period in different projection directions using a biplanar x-ray device in an inflow phase and/or an outflow phase of the contrast agent by back projection of the images. A three-dimensional reconstruction dataset is determined from the projection of the images depending on the flow information during a filling phase in which the contrast agent is present evenly distributed in the blood vessel system. The three-dimensional reconstruction dataset is animated in order to determine the angiography dataset. The capture time periods for the inflow phase and/or the outflow phase are determined from images captured of a test bolus.

### CROSS REFERENCE TO RELATED APPLICATIONS

This application claims priority of German application No. 10 2012 203 751.9 filed Mar. 9, 2012, which is incorporated by reference herein in its entirety.

### FIELD OF INVENTION

The application relates to a method for determining a four-dimensional angiography dataset describing the flow of contrast agent over time through at least one blood vessel system of the body of a patient. In addition, the application relates to a biplanar x-ray device.

### BACKGROUND OF INVENTION

Digital subtraction angiography, DSA for short, is an image capture technology already widely known in the prior art. In this case, images are captured of a vascular system of a patient to whom a contrast agent has previously been administered, such that the vessels filled with contrast agent can be identified well on said images. If from said images obtained using contrast agent (frequently also called “fill images”) mask images are taken off which were captured with the patient in the same position prior to the presence of contrast agent in the vascular system to be imaged, apart from noise effects what remain are merely the signal components of the contrast agent, which means that an excellent assessment of the resulting DSA images is possible.

In this situation, DSA is used not only in cases in which it is a question of a basic mapping or a basic assessment of the vascular structure of a patient but also if the spread of the contrast agent is currently to be investigated. In this situation, diagnostic relevance are cases which show a clearly too slow or clearly too fast spread of the contrast agent in a blood vessel or vessel section. Such effects can be indications of a stenosis or an illness in which the blood can enter the veins at too high a pressure. Such types of examinations are frequently carried out in the region of the brain of a patient.

An established examination method for cases in which the spread of the contrast agent is to be investigated is two-dimensional time-dependent DSA because two-dimensional images can be captured sufficiently quickly in succession in order to achieve an adequate time resolution with respect to the spread of the contrast agent. The capture of three-dimensional image datasets of the target area takes quite a long time which means that, for example when using an x-ray device having a C-arm, it is not possible to sufficiently quickly obtain the time-dependent information in adequately time-resolved fashion. Accordingly, the time-resolved, spatially three-dimensional DSA, which is generally referred to as four-dimensional DSA, is generally used in cases in which for example the movement of the heart is to be observed on the basis of images captured during a filling phase in which the contrast agent is present evenly distributed in the vascular system. In this situation, it is for example known to assign the captured two-dimensional projection images to different movement phases of the heart and to produce for each movement phase individual three-dimensional reconstruction datasets from the projection images, which are then combined to form a moving, in other words animated, image dataset covering a heart phase.

If however the contrast agent inflow and/or the contrast agent outflow is to be observed in a blood vessel system, in a vascular system of the brain, hitherto only time-dependent two-dimensional DSA has been used, which has the disadvantage that in a set projection direction vessels can overlay one another in such a manner that on the one hand the assignment of the information in the three-dimensional vascular tree is rendered more difficult but on the other hand it is also possible that some effects cannot be observed at all because they are obscured by the further vessels lying above or below in the projection direction.

US 2011/0 038 517 A1 relates to a system and a method for four-dimensional angiography and fluoroscopy. There it is proposed to also capture a time series of two-dimensional images in addition to a three-dimensional image of a subject, wherein the time series of the two-dimensional images is to be selectively combined with the three-dimensional image in order to produce four-dimensional images. In this situation, digital subtraction angiography techniques can be employed. It is proposed to use a biplanar system in order to capture two-dimensional images in the time series using different projections, wherein the projection directions can be situated at an angle of 90° with respect to one another.

An image processing device and a method for blood flow imaging are known from US 2008/0 192 997 A1. In this situation, a time series of three-dimensional images which show the blood flow in a vascular tree of an object is to be produced. The application disclosed therein is based on the idea that timing information concerning the blood flow (or the flow of contrast agent) in the vascular tree is obtained from two different projection directions and is mapped onto a three-dimensional volume, where a three-dimensional volume image is produced from a first series of x-ray projection images, timing information can be derived from second and third series of x-ray projection images.

### SUMMARY OF INVENTION

The object of the application is to determine with minimum effort and in a simple manner a four-dimensional angiography dataset showing the temporal progress of the flow of contrast agent in a blood vessel system and containing all three spatial dimensions.

The object is achieved by a method according to the features described in the claims.

In this situation, it should firstly be noted that the administration of the contrast agent itself and the manner in which it enters the vascular system are not part of the present application. Said application relates to an imaging and image evaluation method and the combination of information from different image capture phases.

According to the application, it is proposed to use a biplanar x-ray device. Such types of x-ray devices are already known in the prior art and have two radiographic arrangements each having an x-ray source and an x-ray detector, which can be arranged for example on C-arms which can be moved independently and/or jointly. It has been recognized that such a biplanar x-ray device makes it possible during the inflow phase and the outflow phase of the contrast agent to simultaneously capture two-dimensional images, which according to the phase can be referred to as inflow images and outflow images, in various projection directions, in other words using various projection angles. In this situation the projection directions of the radiographic arrangements are oriented perpendicularly to one another. From such two-dimensional inflow and/or outflow images captured using different projection directions at the same point in time it is however possible by back projection to also determine three-dimensional positional information of image data showing the presence of contrast agent. Ideally, if the temporal progress is observed at back-projected locations, this results in four-dimensional flow information for the various blood vessels of the vascular system which indicates the extent to which the blood vessel is already or still filled with contrast agent.

After the capture of inflow images or prior to the capture of outflow images, projection images are however also captured using joint rotation of the radiographic arrangements in a filling phase in which the contrast agent bolus extends over the entire vascular system to be examined, in which all the vessels consequently contain contrast agent. In this situation, each of the radiographic arrangements captures half of the projection images, which means that it is possible to significantly more quickly actually capture sufficient images in the narrow time window of the filling phase in order to enable a high-resolution reconstruction as artifact-free as possible of a three-dimensional reconstruction dataset, for example using the method of filtered back projection.

In this situation it should be noted at this point that the method according to the application can be applied to blood vessel systems which are moved less by the heartbeat, such as in comparison with the heart region, with the result that there is no need to differentiate between different movement phases.

As is fundamentally known, provision can be made that the projection directions for the projection images are chosen such that a projection angle interval of 180° plus the fan angle of the radiographic arrangements is covered. Under this condition, an analytical reconstruction of a three-dimensional image dataset is possible according to the theory underlying filtered back projection.

After image capture has taken place, in the method according to the application at least two different sets of images are present, namely an inflow image set and/or an outflow image set and also a projection image set. By the projection image set, a high-resolution largely artifact-free representation of the vascular system can be generated in a three-dimensional reconstruction dataset which contains the information about it which can also be generated numerically for example by segmentation algorithms, where a vessel of the vascular system is present in the three-dimensional space. If, as explained above, the four-dimensional flow information is generated from the inflow image set and/or the outflow image set, then the “fill levels” of the vessels given therein can be transferred on account of the precise knowledge of the three-dimensional structure of the vascular system into the three-dimensional reconstruction dataset, and indeed at any point in time. The result is a four-dimensional angiography dataset, which means a three-dimensional volume at any point in time during the entire inflow and/or the entire outflow. Such a four-dimensional angiography dataset is however an excellent starting point for the diagnostic assessment of the blood flow or other phenomena of interest within the examined vascular system of the patient. In this situation, the method according to the application demonstrates for the first time a way of determining the temporal progress of the contrast agent inflow and/or the contrast agent outflow with high resolution in a three-dimensional volume.

In this situation, it is naturally expedient in the context of the present application if digital subtraction angiography is employed in order to obtain the flow information and the reconstruction dataset. In this manner it is possible to extract solely the pixels or voxels (image elements) containing contrast agent. In this situation, provision can specifically be made that as a result of time-resolved capture of two-dimensional images using both radiographic arrangements and subtraction of mask images captured in the same capture geometry with the patient in the same position two-dimensional inflow images of an inflow image set are determined during the inflow phase of the contrast agent, and/or two-dimensional outflow images of an outflow image set are determined during the outflow phase of the contrast agent from the vascular system, and during the filling phase with joint rotation of both radiographic arrangements the projection images are captured using different projection angles, and from these are determined reconstruction images through subtraction of mask images captured in a mask run in the same capture geometry, whereupon the three-dimensional reconstruction dataset is determined from the reconstruction images. Such procedures for the capture of DSA images are fundamentally already known in the prior art. It is important in this case that the patient is moved as little as possible between the capture of mask images and the associated images filled with contrast agent, as is also necessary throughout the time the contrast agent bolus is flowing through the vascular system.

Implementable directly in the context of digital subtraction angiography, but conceivable in principle by way of threshold values also without a subtraction, it is advantageous if the images and the reconstruction dataset are considered in binary fashion, wherein the one value describes the presence of contrast agent in an image element and the other value describes the absence of contrast agent in an image element. In this manner, images which are simple to interpret are given, which can still be overlaid for example with a model of the vascular system produced by segmentation from the three-dimensional reconstruction dataset with regard to the temporal representation of the inflow phase and/or the outflow phase. However, binary image data means that a simple linkage is also enabled of the three-dimensional flow information at a point in time with the image data of the three-dimensional reconstruction dataset. Provision can be made that the fill information at any point in time for each image element of the reconstruction dataset indicating contrast agent includes an associated binary value indicating the presence of contrast agent, with the animation taking place through multiplication at points in time of the image data of the reconstruction dataset with the associated binary values. As a result of back projection in the inflow images or outflow images and the three-dimensional location of image elements indicating contrast agent obtained, a binary value which specifies whether or not at the point in time associated with the binary value contrast agent was present at the location of the image element can be assigned to each image element of the reconstruction dataset indicating the presence of contrast agent and comprising image data. A simple multiplication enables the linkage of image data and binary values, such that the three-dimensional reconstruction dataset is suitable for any point in time and can be modified, and the animated, four-dimensional angiography dataset results from series connection of the thus modified three-dimensional reconstruction datasets.

In an embodiment of the present application, provision can be made that the two-dimensional images are captured in the inflow phase and/or the outflow phase during a joint rotation of the radiographic arrangements through an angle. Provision can also already be made during the capture of inflow and/or outflow images that the projection directions are changed through joint rotation of the radiographic arrangement. This is based on the idea that it may well be the case in certain projection directions that vessels or vessel sections are superimposed in at least one two-dimensional image, which can make it more difficult to determine and to assign the three-dimensional information. If a certain projection angle interval is now cycled through, at various points in time different views of such an overlap region are captured in which it is possible to recognize the different vessels or vessel sections. This in turn makes it possible by using suitable algorithms to identify vessels or vessel sections not visible at times and to extrapolate or interpolate flow information, for example binary values for the periods of time in which the vessel or the vessel section was not visible as a result of superimposition. This once again clearly increases the quality of the flow information because incorrect assignments are minimized and flow information can also be produced by suitable extrapolation and/or interpolation for vessels or vessel sections not visible as a result of superimposition in some two-dimensional images.

Also relevant in the context of the present method is the passage of time of the captured images because this needs to be correlated, or synchronized, with the progress of the contrast agent bolus within the vascular system. Here a plurality of possibilities is already fundamentally known from the prior art for automatically and/or manually determining and accordingly applying capture time periods during the inflow phase, the filling phase and the outflow phase.

Provision is made according to the application that the capture time periods for the inflow phase and/or the outflow phase and/or the filling phase are determined from images captured of a test bolus automatically. In the context of a test bolus measurement, such as two-dimensional test bolus images are captured in this situation, from which can be produced for example time contrast curves for the artery principally leading into the vascular system to be observed and the vein principally leading out which describe the arrival time of the contrast agent in the vascular system, the filling phase and the outflow time of the contrast agent from the system. From these it is then possible to determine manually and/or also automatically periods of time after the commencement of contrast agent administration in which inflow images and/or outflow images and also projection images can be captured. It is also possible to automatically start the capture operations by a control unit of the x-ray device depending on the test bolus information.

Expediently in the context of the present application it is however also conceivable that in order to monitor the spread of the main bolus two-dimensional fluoroscopy or DSA images are captured which are displayed and/or automatically evaluated. If for example it is merely to be observed whether the contrast agent bolus reaches the blood vessel system and/or begins to flow away again from the blood vessel system, it may be sufficient to capture two-dimensional fluoroscopy or DSA images at a low dose, whereupon if contrast agent becomes visible in the vascular system or if contrast agent in the vascular system begins to disappear from the image, the measurement can then be commenced manually and/or automatically, by then switching over to a higher-resolution capture mode and/or by initiating a joint rotation of the radiographic arrangements with regard to joint image capture.

As already explained, the method according to the application is also suitable for imaging a blood vessel system of the brain of the patient. Pathologies which can result in veins becoming overloaded or in too small a supply to certain areas of the brain can be detected and assessed in the three-dimensional space by using the four-dimensional angiography dataset in a subsequent diagnosis. In this situation, it should be noted at this point that it is fundamentally also conceivable in the method according to the application to automatically undertake a quantitative evaluation of the four-dimensional angiography dataset, for example with regard to flow rates through individual vessels or vessel sections of the vascular system and the like. Such automatic evaluation operations, for example with regard to the cerebral blood flow, are fundamentally already known in the prior art but in the context of the present application can also be carried out on the basis of a dimensional angiography dataset describing the variation with time in high resolution in the three-dimensional space.

In addition to the method, the application also relates to a biplanar x-ray device, comprising two radiographic arrangements each having a radiation source and a radiation detector and a control unit, which are oriented or can be oriented perpendicularly to one another in different projection directions, which x-ray device is designed so as to carry out the method according to the application. The capture and evaluation steps of the method according to the application can be implemented by software and/or hardware components in a control unit of a biplanar x-ray device as a computing device, which means that the biplanar x-ray device is extended by the functionality according to the application. All statements relating to the method according to the application can be applied by analogy to the biplanar x-ray device according to the application which enables the feature of the present application to likewise be obtained.

### BRIEF DESCRIPTION OF THE DRAWINGS

Details of the present application will emerge from the embodiments described in the following and with reference to the drawings. In the drawings:

FIG. 1 shows an x-ray device according to the application,

FIG. 2 a flowchart of the method according to the application,

FIG. 3 shows an illustration relating to a first capture step of the method according to the application,

FIG. 4 shows an illustration relating to a second capture step of the method according to the application and

FIG. 5 shows an illustration relating to a third capture step of the method according to the application.

### DETAILED DESCRIPTION OF INVENTION

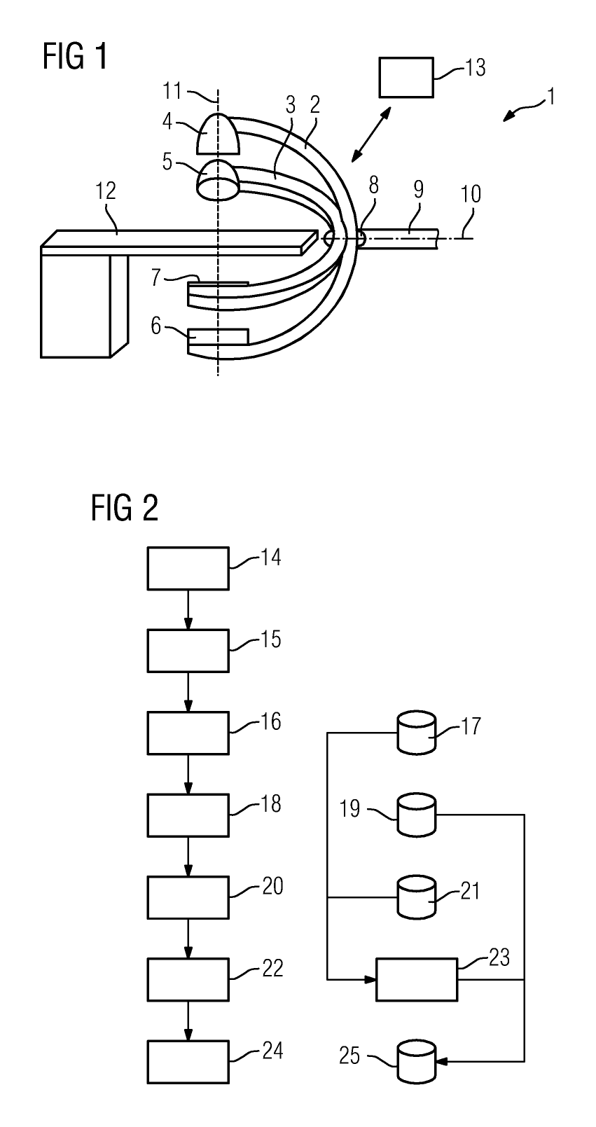

FIG. 1 shows a schematic diagram of an x-ray device 1 according to the application. This comprises two C-arms 2 and 3, on each of which are arranged opposite one another a radiation source 4 and 5 and a radiation detector 6 and 7. In this situation the radiation source 4 and the radiation detector 6 consequently form a first radiographic arrangement, and the radiation source 5 and the radiation detector 7 form a second radiographic arrangement. The C-arms 2 and 3 are connected at the center by way of a joint 8 such that their angular position with respect to one another can on the one hand be changed but on the other hand can also be fixed, for example by way of a locking device on the joint 8. Adjustment of the C-arms is possible by way of the mounting 9, only suggested here, and appropriate drive devices. In this situation, it is possible with regard to the x-ray device 1 to rotate both C-arms 2 and 3 fixed in a certain angular position with respect to one another jointly around an axis 10.

In this situation the radiographic arrangements 4 and 6 and also 5 and 7 move in an imaging plane 11 around a patient table 12 on which a patient to be imaged can be placed. Operation of the x-ray device 1 is controlled by way of a control unit 13, illustrated only schematically, which is designed so as to carry out the method according to the application for determining a four-dimensional angiography dataset.

This method will now be described in detail with regard to FIG. 2. In this situation, it is assumed in the following from the embodiment that the two C-arms 2 and 3 are set up with respect to one another such that the projection directions are perpendicular to one another, but that purely fundamentally other relative positions of the C-arms 2 and 3 with respect to one another are also possible.

Firstly in a step 14, test bolus images are captured and evaluated in order to determine in temporal terms for a main bolus an inflow phase of an administered contrast agent, a filling phase in which the contrast agent is present evenly distributed in a vascular system to be imaged and an outflow phase of the contrast agent from the blood vessel system. To this end, the patient in whom in the present embodiment a blood vessel system of the brain is to be examined is placed on the patient table 12 and after administration of the test bolus two-dimensional fluoroscopy or DSA images are captured, from which are extracted manually or automatically contrast agent progress charts which are in turn likewise evaluated manually or automatically for the purpose of determining ideal capture time periods for observation of the inflow, the filling phase and the outflow. In this situation, the periods of time are determined such that the entire inflow operation and the entire outflow operation can be recorded on images in the following.

It is also conceivable in the context of the method according to the application after administration of the main bolus to constantly produce two-dimensional DSA images or fluoroscopy images of the vascular system, from which the commencement of the inflow and the commencement of the outflow are apparent. These DSA images, or fluoroscopy images, which in the present case can be captured using one or both radiographic arrangements and are produced at a low dose, are observed by a user or else evaluated automatically in order to determine the commencement of the inflow phase or of the outflow phase and also to recognize the filling phase. Accordingly, the measurements discussed in more detail in the following are then started.

In a step 15, mask runs are carried out which means that for all the images yet to be captured in which contrast agent of the main bolus is present mask images are now captured with the patient already in exactly the same position, which as is fundamentally known in digital subtraction angiography can then be subtracted from the images captured with contrast agent.

After the contrast agent of the main bolus is administered, in the inflow phase in step 16 a first capture time period of the present application then commences, the result of which is a set of two-dimensional inflow images. In this situation, the inflow images are captured simultaneously by both radiographic arrangements, which means that at each moment of capture two x-ray images of the blood vessel system captured in projection directions perpendicular to one another are obtained, from which are obtained through subtraction of the corresponding mask images, as known in the prior art, inflow images which depict the presence of contrast agent in the blood vessel system. In the present case 15 images per second are captured here, which means that given a duration of the inflow phase of for example 5 seconds 75 time steps result.

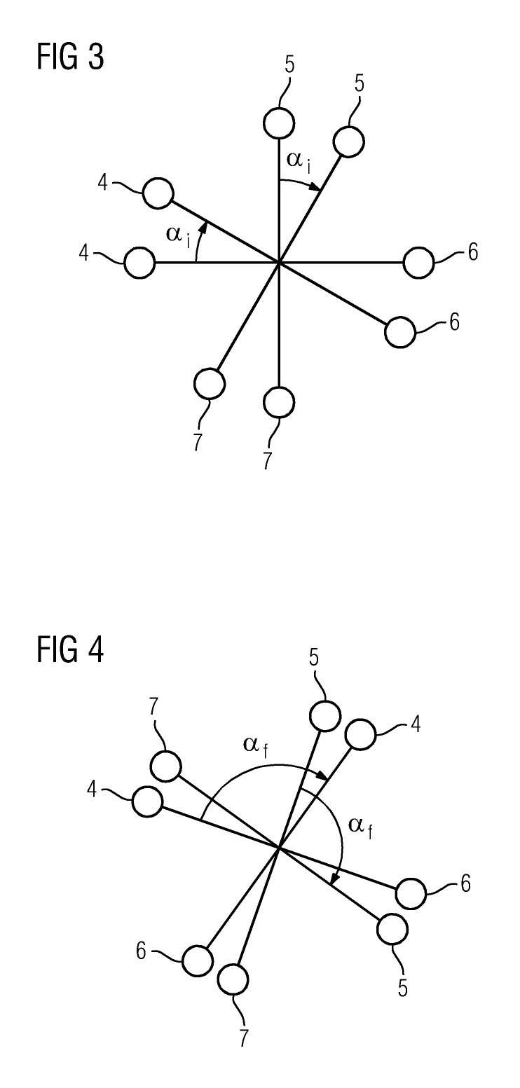

In this situation, the radiographic arrangements are rotated simultaneously during the capture of the inflow images, in the present case during the capture period through an angle αᵢ, which is illustrated by FIG. 3. This serves to ensure that for projections in which blood vessels or vessel sections are superimposed, projections exist at other points in time in which this superimposition is overridden.

The result of step 16 is an inflow image set 17 indicated schematically in FIG. 2, which is initially saved.

Step 16 can be followed by a pause in capture, which is not fundamentally necessary but is conceivable since on account of using both radiographic arrangements a significant time saving, a halving of the capture time, is also given in the step 18 which then follows.

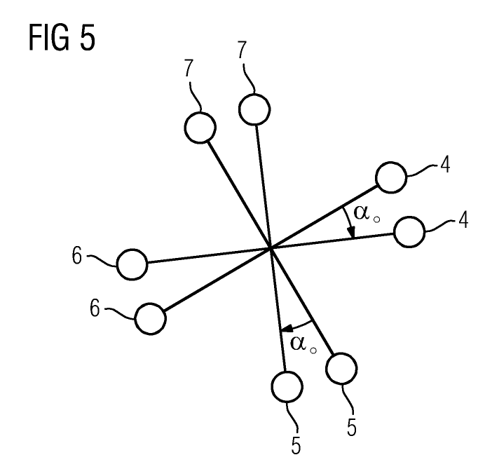

In step 18, during the filling phase two-dimensional projection images are then namely captured during a joint rotation of the radiographic arrangements around the target area. In this situation, the angle α_{f} through which the radiographic arrangements continue to be jointly rotated is chosen such that 180° plus the fan angle of the radiographic arrangements are covered as the projection angle interval, which means that a reconstruction which is as artifact-free as possible becomes possible, for example by filtered back projection. This is represented schematically by FIG. 4.

The corresponding mask images from step 15 are also subtracted from the projection images and two-dimensional reconstruction images are produced from which in the present case, for example by filtered back projection, a three-dimensional reconstruction dataset 19 is determined which clearly shows the blood vessel system (since it is filled with contrast agent).

In this situation it can moreover be expedient to determine the reconstruction dataset using binary image data, wherein one possible value indicates the presence of contrast agents in the voxel but a further possible value specifies that no contrast agent is present in the voxel. It is furthermore expedient to segment the vascular system in the reconstruction dataset 19 and for example to hold available the boundaries of the vessels for later presentation purposes.

In a step 20, a further capture time period during the outflow phase of the contrast agent from the vascular system is used by analogy with step 18 in the present case in order to determine an outflow image set 21, wherein the same capture frequency is used. If the outflow phase lasts exactly as long as the inflow phase, thus in the example five seconds, then two two-dimensional outflow images captured in projection directions perpendicular to one another are again present in each case at 75 time steps.

A joint rotation of the radiographic arrangement, here through an angle αₒ, also takes place during the capture of the outflow images in step 18. A corresponding schematic illustration can be seen in FIG. 5.

The inflow images or outflow images from a point in time standing perpendicular to one another are then suitable for back-projecting a three-dimensional position for displayed pixels filled with contrast agent. This means that at any point in time the two two-dimensional images standing perpendicular to one another are evaluated in order to determine the three-dimensional position of vessels or vessel sections already to be seen thereon or still filled with contrast agent. This happens in step 22. At any point in time, information thus then exists associated with voxels filled with contrast agent of the reconstruction dataset 19 about whether contrast agent was already or still present at this position. The flow information 23 determined thus specifies in three-dimensional and time-dependent fashion where contrast agent was already or still present in the vascular system, which for example can be mapped by way of a binary value associated with one or more image elements (voxels) of the reconstruction dataset 19 indicating the presence of contrast agent.

In this situation it should also be noted at this point that sub-steps of step 22 are also concerned with the extrapolation or interpolation of blood vessels or blood vessel sections obscured in some projection directions, or with the advance identification. After imaging has also taken place in steps 16 and 20 using different projection directions, vessels and vessel sections obscured in some projection directions can be identified, assigned and periods of time determined in which no information is present there. It is then possible to interpolate into, or extrapolate into, these periods of time. A dataset covering the inflow phase and the outflow phase is thus determined as flow information 23.

Finally, in a step 24 the reconstruction dataset 19 is animated taking into consideration the flow information 23. This means that modified reconstruction datasets which show the filling state at this point in time are produced for each of the points in time, for example by multiplication of the binary value with the image data of the associated pixels. Modified reconstruction datasets thus result for each point in time which, displayed in succession, yield a four-dimensional, in other words moving, image of the flow of contrast agent in the vascular system, which is stored as a four-dimensional angiography dataset 25. If prior to that one of the edges of the vessels of the vascular system was determined by segmentation as a three-dimensional representation, the latter can be displayed superimposed on the dimensional angiography dataset 25 which means that a user can easily see how the vessels of the blood vessel system are filled up in the inflow phase and how the contrast agent bolus leaves the blood vessel system again in the outflow phase. The four-dimensional angiography dataset thus shows the passage of the contrast agent bolus flowing through the three-dimensional volume of the vascular system over time.

In conclusion it should further be noted that the angles αᵢ and αₒ can be predefined as fixed but it is also conceivable to configure them as selectable by a user, in which case empirically meaningful angles can be used. This also applies to pauses in capture which may be used and the decision as to whether or not the C-arms 2 and 3 continue to be rotated during pauses in capture.

Although the application has been illustrated and described in detail by the embodiment, the application is not restricted by the disclosed examples and other variations can be derived by the person skilled in the art without departing from the scope of protection of the application.

1. A method for determining a four-dimensional angiography dataset describing a flow of contrast agent over time through a blood vessel system of a body of a patient, comprising:
capturing test bolus images for determining a time period;
capturing two-dimensional images in the time period in different projection directions using a biplanar x-ray device in an inflow phase and/or an outflow phase of the contrast agent, wherein the biplanar x-ray device comprises two radiographic arrangements each having a radiation source and a radiation detector;
obtaining a four-dimensional flow information from the two-dimensional images captured in the time period by back projection of the images;
determining a three-dimensional reconstruction dataset from the projection of the images depending on the four-dimensional flow information during a filling phase in which the contrast agent is present evenly distributed in the blood vessel system; and
animating the reconstruction dataset for determining the angiography dataset.

2. The method as claimed in claim 1, further comprising capturing mask images in a mask run and digital angiography subtracting based on the mask images for obtaining the flow information and the reconstruction dataset.

3. The method as claimed in claim 2, further comprising:
determining two-dimensional inflow images during the inflow phase of the contrast agent;
determining two-dimensional outflow images during the outflow phase of the contrast agent;
determining reconstruction images during the filling phase by subtracting the mask images in same capture geometry; and
determining the reconstruction dataset from the reconstruction images.

4. The method as claimed in claim 1, wherein the two-dimensional images and the reconstruction dataset are binary value, wherein one value of the binary value indicates presence of the contrast agent in an image element and another value of the binary value indicates absence of the contrast agent in the image element.

5. The method as claimed in claim 4, wherein the flow information at any point in the time period for the image element of the reconstruction dataset comprises the one value indicating the presence of the contrast agent, and wherein the reconstruction dataset is animated through multiplying image data of the reconstruction dataset at points in the time period with the one value.

6. The method as claimed in claim 1, wherein the two-dimensional images are captured in the inflow phase and/or the outflow phase during a joint rotation of the radiographic arrangements through an angle.

7. The method as claimed in claim 1, wherein the projection directions cover a projection angle interval of 180° plus a fan angle of the radiographic arrangements.

8. The method as claimed in claim 1, wherein the test bolus images are two-dimensional fluoroscopy or digital subtraction angiography images for monitoring spread of the test bolus, and wherein the test bolus images are displayed and/or automatically evaluated.

9. The method as claimed in claim 1, wherein the blood vessel system comprises a vascular system of a brain of the patient.

10. A biplanar x-ray device, comprising:
two radiographic arrangements oriented in different projection directions each having a radiation source and a radiation detector; and
a control unit adapted to perform a method as claimed in claim 1.
